(19) United States
(12) Patent Application Publication
(10) Pub. No.: US 2026/0022340 A1
(43) Pub. Date: Jan. 22, 2026

(54) ATRIOVENTRICULAR NODE-LIKE PACEMAKER CELLS

(71) Applicant: UNIVERSITY HEALTH NETWORK, Toronto (CA)

(72) Inventors: Stephanie I. PROTZE, Toronto (CA); Michelle LOHBIHLER, Toronto (CA)

(21) Appl. No.: 18/993,925

(22) PCT Filed: Jul. 12, 2023
(86) PCT No.: PCT/CA2023/050936

§ 371 Date: Jan. 13, 2025

Related U.S. Application Data
(60) Provisional application No. 63/368,250, filed on Jul. 12, 2022.
(60) Provisional application No. 63/378,031, filed on Sep. 30, 2022.

(51) Int. Cl. C12N 5/077; A61K 35/34; A61P 9/00; C12N 5/074

(52) U.S. Cl. CPC C12N 5/0657; A61K 35/34; A61P 9/00; C12N 5/0696; C12N 2501/115; C12N 2501/155; C12N 2501/16; C12N 2501/165; C12N 2501/415; C12N 2501/727

(57) ABSTRACT

The present disclosure provides methods of generating atrioventricular node like pacemaker cells in tissue culture and methods of using these cells.

### RELATED APPLICATIONS

This Patent Cooperation Treaty application claims the benefit of priority of U.S. Provisional Application No. 63/368,250 filed on Jul. 12, 2022, and U.S. Provisional Patent Application No. 63/378,031 filed on Sep. 30, 2022, which are incorporated herein in their entirety.

### BACKGROUND OF THE INVENTION

A healthy human heart experiences over 3 billion beats in a typical lifetime owing to the remarkable capacity of the pacemaker cells to produce rhythmic and synchronized contractions of the upper (atrial) and lower (ventricular) chambers (Christoffels and Moorman, Circ Arrhythm Electrophysiol. (2009) 2(2):195-207). The Sinoatrial Node (SAN) spontaneously generates the electric impulse (Boyett et al., Cardiovasc Res. (2000) 47(4):658-687) that propagates through the atria to the Atrioventricular Node (AVN) that establishes and coordinates the connection between the chambers (Bakker et al., Trends Cardiovasc Med. (2010) 20(5):164-71). Block of impulse propagation at the AVN causes heart block (AV-block) and can result in significant adverse symptoms including syncope and cardiac death.

Due to the poor regenerative capacity of the heart, the current standard of care for AV-block is lifelong electronic pacemaker (EPM) implantation (Epstein et al., (2008) Heart Rhythm 5(6):e1-62). In Canada 21,000 EPM devices are implanted each year, and although effective, entail substantial risks. EPMs lack autonomic responsiveness and have a relatively high complication rate (˜16%) caused by thoracic trauma, lead complication and infection (Cantillon et al., JACC Clin Electrophysiol. (2017) 3:1296-1305; Kirkfeldt et al., Eur Heart J. (2014) 35:1186-94; Udo et al., Heart Rhythm (2012)9:728-35). Pediatric patients are especially impacted by battery replacements every 5-10 years and lead refitting surgeries due to the lack of growth adaptation. Thus, the ideal replacement for a damaged AVN would be a biological conduction bridge, that can propagate the electrical impulses like the AVN.

Human pluripotent stem cells (hPSCs) that can be differentiated in vitro into all cell types that closely resemble the endogenous counterparts represent an ideal cell source for a biological conduction bridge. In the past decade, protocols have been defined to differentiate hPSCs into cardiomyocytes (Laflamme et al., Nat Biotechnol. (2007) 25(9):1015-24; Yang et al., Nature (2008) 453(7194):524-8; Kattman et al., Cell Stem Cell (2011) 8(2):228-40). In particular, a method was described to differentiate hPSCs into SAN-like pacemaker cells (SANLPCs) that were able to pace host tissue and function as biological pacemaker when transplanted into rat hearts (Protze et al., Nat Biotechnol. (2017) 35(1):56-68). Importantly SAN and AVN pacemaker cells have distinct phenotype and function, therefore to treat diseases of the AVN hPSC-derived AVN-like pacemaker cells (AVNLPCs) are required.

Thus, there remains a need for methods of generating and using AVNLPCs to restore the electrical activity of a diseased heart.

### SUMMARY OF THE INVENTION

In an aspect, the present disclosure provides methods of generating a population of cardiomyocytes enriched for atrioventricular node-like pacemaker cells (AVNLPCs), which comprises: (a) providing a starting population of human cardiac progenitors; and (b) culturing the human cardiac progenitors in a medium comprising a Wnt agonist or a bone morphogenetic protein (BMP) component to generate AVNLPCs.

In some embodiments, methods of generating a population of cardiomyocytes enriched for atrioventricular node-like pacemaker cells (AVNLPCs) comprise (a) providing a starting population of human cardiac progenitors; and (b) culturing the human cardiac progenitors in a medium comprising a Wnt agonist to generate AVNLPCs.

In some embodiments, methods of generating a population of cardiomyocytes enriched for atrioventricular node-like pacemaker cells (AVNLPCs) comprise (a) providing a starting population of human cardiac progenitors; and (b) culturing the human cardiac progenitors in a medium comprising a bone morphogenetic protein (BMP) component to generate AVNLPCs.

In other embodiments, step b) comprises culturing the human cardiac progenitors in a medium comprising a bone morphogenetic protein (BMP) component, optionally for about 4 days, and culturing the progenitors in a medium comprising a Wnt agonist, optionally for about 4 to about 5 days, to generate AVNLPCs. In these embodiments, the culturing with the BMP component and Wnt agonist can overlap or partially overlap, for example, at least partially comprise culturing the progenitors in a medium comprising a bone morphogenetic protein (BMP) component and a Wnt agonist, optionally for about 1 day, 2 days, 3 days or more, including wherein the step comprises culturing the progenitors in a medium comprising a BMP component and a Wnt agonist simultaneously.

In some embodiments, the AVNLPCs are characterized by being NKX2-5⁺TBX3⁺.

In some embodiments, the medium further comprises VEGF, optionally at 1-20 ng/ml, further optionally at 5 mg/mL.

In some embodiments, the medium further comprises VEGF.

In some embodiments, the Wnt agonist is CHIR-99021, optionally at 1-20 μM or at any range between 1-20 μM, for example 1, 2, 3, 4, 5, 6, 7, 8, 9, 10, 11, 12, 13, 14, 15, 16, 17, 18, 19, or 20 μM.

In some embodiments, the Wnt agonist is CHIR-99021.

In some embodiments, the medium comprises CHIR-99021 and VEGF, optionally wherein the incubating step is performed for about 1-10 days, optionally about 2-10, 2-8, 3-6, 3-5, or 5-10 days, optionally about 3 or 4 days.

In some embodiments, the medium comprises CHIR-99021 and VEGF.

In some embodiments, the incubating step (e.g., the culturing step) is performed for about 1-10 days, optionally about 2-10, 2-8, 3-6, 3-5, or 5-10 days, optionally about 3 or 4 days.

In some embodiments, the incubating step is performed for about 1-10 days.

In some embodiments, the incubating step is performed for about 2-10, 2-8, 3-6, 3-5, or 5-10 days.

In some embodiments, the incubating step is performed for about 3 or 4 days.

In some embodiments, the BMP component is BMP2, optionally at 10-500 ng/mL.

In some embodiments, the medium comprises BMP2 and VEGF, optionally wherein the incubating step is performed for about 1-10 days, optionally about 2-10, 2-8, 3-6, 3-5, or 5-10 days, optionally about 3 or 4 days.

In some embodiments, the cardiac progenitor cells are obtained by culturing cardiogenic mesoderm cells in the presence of a Wnt inhibitor and VEGF, optionally for about 2 to 7 days.

In some embodiments, the cardiac progenitor cells are obtained by culturing cardiogenic mesoderm cells in the presence of a Wnt inhibitor and VEGF.

In some embodiments, the cardiogenic mesoderm cells are cultured for about 2 to 7 days.

In further embodiments, the cardiac progenitor cells are obtained by culturing cardiogenic mesoderm cells in the presence of a Wnt inhibitor that is IWP2, optionally at 0.5 to 10 μM, and/or VEGF at 1-20 ng/mL.

In some embodiments, the cardiac progenitor cells are obtained by culturing cardiogenic mesoderm cells in the presence of VEGF at 1-20 ng/ml.

In some embodiments, the cardiac progenitor cells are obtained by culturing cardiogenic mesoderm cells, wherein the cardiogenic mesoderm cells are obtained by culturing embryoid bodies in the presence of a cardiac progenitor inducing medium comprising one or more, or all, of BMP component and an activin/Nodal component, FGF, and a Wnt agonist, optionally for about 1, 2, or 3 days. In further embodiments, the cardiac progenitor inducing medium comprises: BMP4, optionally at 1-100 ng/ml, Activin A, optionally 1-100 ng/ml, bFGF, optionally at 1-20 ng/ml), and CHIR-99021, optionally at 1-10 μM.

In some embodiments, the cardiogenic mesoderm cells are obtained by culturing embryoid bodies in the presence of a cardiac progenitor inducing medium comprising all of BMP component and an activin/Nodal component, FGF, and a Wnt agonist, optionally for about 1, 2, or 3 days.

In some embodiments, the cardiogenic mesoderm cells are obtained by culturing embryoid bodies in the presence of a cardiac progenitor inducing medium comprising: BMP4, optionally at 1-100 ng/mL, Activin A, optionally 1-100 ng/ml, bFGF, optionally at 1-20 ng/ml, and CHIR-99021, optionally at about 1-10 μM.

In some embodiments, the cardiogenic mesoderm cells are obtained by culturing embryoid bodies in the presence of a cardiac progenitor inducing medium comprising: BMP4, Activin A, bFGF, and CHIR-99021.

In some embodiments, the cardiogenic mesoderm cells are obtained by culturing embryoid bodies in the presence of a cardiac progenitor inducing medium comprising: BMP4 at 1-100 ng/mL, Activin A at 1-100 ng/mL, bFGF at 1-20 ng/ml, and CHIR-99021 at about 1-10 μM.

In some embodiments, the cardiogenic mesoderm cells are obtained by culturing embryoid bodies in the presence of a cardiac progenitor inducing medium comprising about 3 ng/ml BMP4 and about 2 ng/ml Activin A, optionally wherein the medium further comprises about 5 ng/ml bFGF and/or about 1-10 μM CHIR-99021.

In some embodiments, the cardiogenic mesoderm cells are obtained by culturing embryoid bodies in the presence of a cardiac progenitor inducing medium comprising about 3 ng/ml BMP4 and about 2 ng/ml Activin A, wherein the medium further comprises about 5 ng/ml bFGF.

In some embodiments, the cardiogenic mesoderm cells are obtained by culturing embryoid bodies in the presence of a cardiac progenitor inducing medium comprising about 3 ng/ml BMP4 and about 2 ng/ml Activin A, wherein the medium further comprises about 1-10 μM CHIR-99021.

In some embodiments, the cardiogenic mesoderm cells are obtained by culturing embryoid bodies in the presence of a cardiac progenitor inducing medium comprising about 5 ng/ml BMP4 and about 4 ng/ml Activin A, wherein the medium further comprises about 5 ng/ml bFGF and/or about 1-10 μM CHIR-99021.

In some embodiments, the cardiogenic mesoderm cells are obtained by culturing human pluripotent stem cells (hPSCs) in the presence of 1-10 ng/ml BMP4 (e.g., 3-8 or 3-5 ng/ml) and 1-15 ng/ml (e.g., 2-12, 1-5, or 2-4 ng/ml) Activin A.

In further embodiments, the cardiogenic mesoderm cells are obtained by incubating human pluripotent stem cells (hPSCs) in the presence of about 3 ng/ml BMP4 and about 2 ng/ml Activin A, optionally also in the presence of about 5 ng/ml bFGF and/or 1-10 μM CHIR-99021.

In other embodiments, the cardiogenic mesoderm cells are obtained by incubating hPSCs in the presence of about 5 ng/ml BMP4 and about 4 ng/ml Activin A, optionally also in the presence of about 5 ng/ml bFGF and/or 1-10 μM CHIR-99021.In some embodiments, the embryoid bodies are obtained by incubating human pluripotent stem cells (hPSCs) in the presence of a BMP, optionally BMP2 or BMP4, and Rho-associated protein kinase (ROCK) inhibitor.

In some embodiments, the embryoid bodies are obtained by incubating human pluripotent stem cells (hPSCs) in the presence of a BMP, either BMP2 or BMP4, and Rho-associated protein kinase (ROCK) inhibitor.

In some embodiments, the embryoid bodies are obtained by incubating human pluripotent stem cells (hPSCs) in the presence of BMP2 and Rho-associated protein kinase (ROCK) inhibitor.

In some embodiments, the embryoid bodies are obtained by incubating human pluripotent stem cells (hPSCs) in the presence of BMP4 and Rho-associated protein kinase (ROCK) inhibitor.

In another aspect, the present disclosure provides an AVNLPC-enriched population of cardiomyocytes generated by any method herein disclosed.

In some embodiments, at least 25, 30, 40, 50, 60, 70, 80, or 90% of the cells in the AVNLPC-enriched population of cardiomyocytes are AVNLPCs.

The AVNLPC enriched population of cardiomyocytes can be generated by any method herein disclosed. In another aspect, the present disclosure also provides a pharmaceutical composition comprising an AVNLPC-enriched cell population as herein disclosed and a pharmaceutically acceptable carrier, and/or articles of manufacture (e.g., kits) comprising the AVNLPC enriched population of cardiomyocytes or pharmaceutical composition or reagents for producing the AVNLPC enriched population according to a method described herein.

In some embodiments, the pharmaceutically acceptable carrier is a hydrogel.

In another aspect, also provided herein are methods of treating a human patient in need thereof, comprising administering a pharmaceutical composition as herein disclosed to a patient.

In another aspect, also provided herein are AVNLPC-enriched populations of cardiomyocytes generated by any method herein disclosed or pharmaceutical compositions as herein disclosed for use in treating a human patient in need thereof.

In another aspect, also provided herein are uses of AVNLPC-enriched populations of cardiomyocytes generated by any method herein disclosed for the manufacture of a medicament for treating a human patient in need thereof.

In some embodiments, the human patient has atrioventricular block.

Features which are described in the context of separate aspects and embodiments of the invention may be used together and/or be interchangeable. Similarly, features described in the context of a single embodiment may also be provided separately or in any suitable sub-combination.

Other features, objectives, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIG. 1 is a schematic of an exemplary atrioventricular node-like pacemaker cell (AVNLPC) differentiation protocol. To induce the AVN phenotype, treatment with either BMP2 from day 5-8 or with CHIR-99021 from day 8-11 can be applied. Combination of both BMP2 and CHIR inductions also results in cultures highly enriched in AVNLPCs.

FIG. 2A is a schematic of a double reporter hPSC line showing eGFP expression under the NKX2-5 gene and tdTomato expression under the TBX3 gene.

FIG. 2B is a schematic of reporter genes expressed by different hPSC-derived cardiomyocyte subtypes.

FIG. 2C illustrates a representative flow cytometric analysis of day 20 cardiomyocytes generated from the reporter cell line from FIG. 2A using a standard ventricular cardiomyocyte differentiation protocol. Note the low number of NKX2-5+TBX3+ cells generated with the ventricular differentiation protocol.

FIG. 2D illustrates an exemplary protocol for obtaining various cardiomyocyte lineages from hPSCs.

FIG. 2E illustrates representative flow cytometric analyses of day 4 mesoderm and day 20 myocytes generated under exemplary conditions using media containing 8 ng/ml BMP4 and 12 ng/ml Activin A (8B/12A), 3 ng/ml BMP4 and 2 ng/ml Activin A (3B/2A), and 5 ng/ml BMP4 and 4 ng/ml Activin A (5B/4A) during the primitive streak stage (days 1-3). The media also contained FGF as described herein. The largest proportion of NKX2-5+TBX3+ cells was obtained from mesoderm induced with 5B/4A. Mesoderm induced using 5B/4A contained a large proportion of cells that did not express CD235a nor RALDH2 markers. CD235a is expressed by mesoderm cells that mature into VLCMs, and RALDH2 is expressed by mesoderm cells that mature into ALCMs/SANLPCs.

FIG. 3A illustrates representative flow cytometric analyses of day 20 myocytes after the treatment with 9 uM CHIR at the indicated timepoints. Myocytes are identified as SIRPA+CD90− cells and AVNLPCs are identified as NKX2-5+TBX3+ cells within this myocyte population.

FIG. 3B shows a summarizing bar graph for the percentage of NKX2-5+TBX3+ cardiomyocytes out of the total cell population at day 20 as shown in FIG. 3A. A significant increase in NKX2-5+TBX3+ AVNLPCs was obtained following treatment with 9 uM CHIR from day 8-11, day 11-15 or day 15-18, (n=5). Error bars represent SEM. One-way ANOVA, Bonferroni's post hoc test: *P<0.05, **P<0.01 vs indicated sample.

FIG. 4A illustrates representative flow cytometric analyses of day 20 myocytes after the treatment with the indicated amounts of CHIR from day 8-11. Myocytes are identified as SIRPA+CD90− cells and AVNLPCs are identified as NKX2-5+TBX3+ cells within this myocyte population.

FIG. 4B shows a summarizing bar graph for the percentage of NKX2-5+TBX3+ cardiomyocytes out of the total cell population at day 20 as shown in FIG. 4A. The highest percentage of NKX2-5+TBX3+ AVNLPCs was obtained following treatment with 12-15 uM CHIR from day 8-11, (n=5). Error bars represent SEM. One-way ANOVA, Bonferroni's post hoc test: *P<0.05, **P<0.01 vs indicated sample.

FIG. 5A shows a summarizing bar graph for the percentage of NKX2-5+TBX3+ cardiomyocytes out of the total cell population after the treatment with 50 ng/ml BMP2 at the indicated timepoints. The highest percentage in NKX2-5-TBX3+ AVNLPCs was obtained following treatment with 50 ng/ml BMP2 from day 5-8, (n=5). Error bars represent SEM. One-way ANOVA, Bonferroni's post hoc test: *P<0.05, **P<0.01 vs indicated sample.

FIG. 5B shows a summarizing bar graph for the percentage of NKX2-5+TBX3+ cardiomyocytes out of the total cell population after the treatment with the indicated amounts of BMP2 from day 5-8. The highest percentage in NKX2-5+TBX3− AVNLPCs was obtained following treatment with 200-400 ng/ml BMP2 from day 5-8, (n=5). Error bars represent SEM. One-way ANOVA, Bonferroni's post hoc test: *P<0.05, **P<0.01 vs indicated sample.

FIG. 6 illustrates that NKX2-5+TBX3+ cells have enriched expression of the atrioventricular node (AVN) genes TBX2, MSX2, and BMP2, but not the sinoatrial node (SAN) cell marker SHOX2, as detected by RT-qPCR. NKX2-5+TBX3+ cells express high levels of the pan-cardiomyocyte markers TNNT2 and NKX2.5. The figure shows the expression of various markers in different cardiomyocyte populations. Shown for reference are expression levels of the indicated genes in sinoatrial node-like pacemaker cells (SANLPC; red), atrial-like cardiomyocytes (ALCM; green), and ventricular-like cardiomyocytes (VLCM; blue); n=5. Pan-pacemaker markers have enriched expression in the NKX2-5+TBX3+ AVNLPCs and SANPLCs. Expression values are normalized to the housekeeping gene TBP. One-way ANOVA, Bonferroni's post hoc test: *P<0.05, **P<0.01 vs indicated sample.

FIG. 7 illustrates representative patch-clamp electrophysiology recordings showing action potentials in NKX2-5+TBX3+ AVNLPCs and NKX2-5+TBX3− VLCMs; n=10 cells each; t-test: **P<0.01, *P<0.05 vs VLCM. AP, action potential; APD90, AP duration at 90% of repolarization; bpm, beats per minute; dv/dtmax, maximum AP upstroke velocity.

FIG. 8 is a schematic illustrating key functions of the AVN in vivo; cm/s (centimeters/second).

FIG. 9 illustrates a strategy for using AVNLPCs as a biological pacemaker that can bridge electrical conduction between the atria and ventricles of the heart.

FIG. 10A shows a representative image of a 3D tissue strip containing cardiac fibroblasts (25%) and NKX2-5+TBX3+ AVNLPCs (75%).

FIG. 10B shows conduction velocities of engineered tissues containing AVNLPCs (eAVNT) and VLCMs (eVT) (n=5); t-test: **P<0.01.

FIG. 10C shows capture ratios of the bottom parts of the eAVNT and eVT tissues following pacing with increasing frequencies at the top. Note that eAVNTs are able to block the conduction of fast rates of 10 Hz by going into a 3:1 conduction block (n=5).

FIG. 11 illustrates an experimental setup for ex vivo electrocardiogramand optical mapping analysis of AVNLPCs implanted in vivo and engrafted between the atria and ventricle of the intact Guinea pig heart, and after inducing acute atrioventricular (AV) block.

FIG. 12 illustrates that the AVNLPCs grafts are insulated from the host heart myocardium; cTNNT indicates host myocardium, Ku80 indicates grafted AVNLPCs, and DAPI indicates all cells.

FIG. 13 illustrates a representative section of a guinea pig heart with removed epicardium using 4-minute Trypsin treatment (right) and a section of a guinea pig heart with intact epicardium (control; left).

FIG. 14 is a schematic showing a model for testing conduction via the AVNLPC graft; AVNLPCs are injected in the border region (light gray outline) of cryoablation-induced scar tissue (dark gray patch) to induce the electrical coupling of AVNLPCs and the host heart tissue, which can then be measured ex vivo.

FIG. 15 is a schematic showing the use of the genetically encoded voltage sensitive dye ASAP1 to assess electrical integration of the graft cells by optical mapping. Human PSCs expressing the genetically encoded voltage sensor ASAP1 were differentiated into AVNLPCs. The voltage change across the cell membrane associated with action potentials (APs) is detected by the voltage sensitive domain (VSD) of the sensor, which alters the fluorescent properties a fused GFP molecule; APs produce a decrease in fluorescence.

FIG. 16 illustrates a host heart with tissue graft (left) and representative recordings of ASAP1 and RH237 voltage indicators showing 1:1 electrophysiological coupling between graft and heart (right); action potentials (APs).

FIG. 17 illustrates a host heart with tissue graft (left) and recordings of ASAP1 and RH237 voltage indicators showing electrophysiologically uncoupled graft and host heart (right); action potentials (APs).

FIG. 18 illustrates AVNLPC injected cell graft located within the border zone of a cryoinjury-induced scar (outlined by dotted white line) and representative recordings of ASAP1 and RH237 voltage indicators showing 1:1 electrophysiological coupling between engrafted cells and host heart; action potentials (APs).

FIG. 19 illustrates maximum capture rates for in vivo AVNLPC and VLCM grafts injected into the border zone of a cryoablation-induced scar. Note that AVNLPC grafts can block conduction of fast rates of 6 Hz by going into a 2:1 conduction block. Error bars represent SEM (n=2).

### DETAILED DESCRIPTION OF THE INVENTION

The present disclosure provides methods of generating a cardiomyocyte (CM) population enriched for AVNLPC cells, compositions comprising AVNLPCs, and methods of administering a composition of AVNLPCs to a subject with a defect in the atrioventricular node (e.g., a subject suffering from an AV block). Naturally occurring atrioventricular node (AVN) cells are limited in numbers, cannot be easily isolated and have low viability. In contrast to naturally-occurring isolated AVN cells harvested from adult heart (i.e., non-fetal cells), AVNLPCs generated using the herein disclosed methods are numerous and remain viable in culture (e.g., at least one week or up to 10 days) and long enough and in great enough numbers to be used, for example, in engraftment procedures or testing. In some embodiments, the AVNLPC cells are identified by double positivity (NKX2-5+TBX3+) for NK2 homeobox 5 (aka. NKX2-5 or NKX2.5) and T-box transcription factor 3 (aka. TBX-3 or TBX3). Previous methods for generating CMs in tissue culture lead to a mixture of CM subtypes, including some pacemaker cells and atrial myocytes; but the majority of the culture has a ventricular phenotype. By contrast, the methods herein generate CM populations in which at least a quarter of the cells (e.g., at least 30, 35, 40, 45, 50, 55, 60, 65, 70, 75, 80, 85, 90%, 95%, or 98%) are AVNLPC cells, as indicated by, e.g., double positivity by NKX2-5 and TBX-3.

The enriched AVNLPC cell populations are advantageous in cardiac regenerative therapy in which the patient's heart requires repair or restoration of atrioventricular node pacemaker functions. Therapeutic compositions comprising such cell populations are expected to closely mimic the functional properties of AVN cells and as such prevent the conduction of fast atrial rhythms to the ventricles where they would be life threatening.

### I. Generation of AVNLPCs in Vitro

AVN pacemaker cells are a type of cardiomyocytes. Cardiomyocytes are contractile cells characterized by the expression of signal-regulatory protein alpha (SIRPA) and one or more of cardiac troponins (e.g., cardiac troponin I or cardiac troponin T (“cTNT”)). Cardiomyocytes are also CD90 negative. Developmentally, cardiomyocyte progenitor cells can be derived from cardiac mesodermal cells (aka cardiovascular mesoderm or mesodermal cells) and will upregulate the expression of cardiomyocyte markers cTNT and NKX2-5 between days 6-20 of differentiation. Mesodermal cells can be PDGFR-alpha positive (e.g., can be denoted as PDGFRa⁺). AVN pacemaker cells are TBX3⁺NKX2-5⁺.

A variety of cell types may be used as a source of cells for the in vitro (including ex vivo) generation of cardiomyocytes such as atrioventricular node-like pacemaker cells (AVNLPCs). The source cells may be, for example, human pluripotent stem cells (PSCs). As used herein, the term “pluripotent” or “pluripotency” refers to the capacity of a cell to self-renew and to differentiate into cells of any of the three germ layers: endoderm, mesoderm, or ectoderm. “Pluripotent stem cells” or “PSCs” include, for example, embryonic stem cells, PSCs derived by somatic cell nuclear transfer, and induced PSCs (iPSCs). As used herein, the term “embryonic stem cells,” “ES cells,” or “ESCs” refers to pluripotent stem cells obtained from early embryos; in some embodiments, this term refers to ES cells obtained from a previously established ES cell line and excludes stem cells obtained by recent destruction of a human embryo.

One convenient source of cells for generating cardiomyocytes such as AVNLPCs is iPSCs. iPSCs are a type of pluripotent stem cell artificially generated from a non-pluripotent cell, such as an adult somatic cell or a partially differentiated cell or terminally differentiated cell (e.g., a fibroblast, a cell of hematopoietic lineage, a myocyte, a neuron, an epidermal cell, or the like), by introducing to the cell or contacting the cell with one or more reprogramming factors. Methods of producing iPSCs are known in the art and include, for example, inducing expression of one or more genes (e.g., POU5F1/OCT4 (Gene ID: 5460) in combination with, but not restricted to, SOX2 (Gene ID: 6657), KLF4 (Gene ID: 9314), c-MYC (Gene ID: 4609), NANOG (Gene ID: 79923), and/or LIN28/LIN28A (Gene ID: 79727)). Reprogramming factors may be delivered by various means (e.g., viral, non-viral, RNA, DNA, or protein delivery); alternatively, endogenous genes may be activated by using, e.g., CRISPR and other gene editing tools, to reprogram non-pluripotent cells into PSCs.

Exemplary methods of isolating and maintaining PSCs, including ESCs and iPSCs, are described in, e.g., Thomson et al., Science (1998) 282(5391):1145-7; Hovatta et al., Human Reprod. (2003) 18(7):1404-09; Ludwig et al., Nat Methods (2006) 3:637-46; Kennedy et al., Blood (2007) 109:2679-87; Chen et al., Nat Methods (2011) 8:424-9; and Wang et al., Stem Cell Res. (2013) 11(3):1103-16.

Exemplary methods for inducing differentiation of PSCs into cells of various lineages are known. For example, numerous methods exist for differentiating PSCs into cardiomyocytes, as shown in, e.g., Kattman et al., Cell Stem Cell (2011) 8(2):228-40; Burridge et al., Nat Protocols (2014) 11(8):855-60; Burridge et al., PLoS ONE (2011) 6:e18293; Lian et al., PNAS. (2012) 109:e1848-57; Ma et al., Am J Physiol Heart Circ Physiol. (2011) 301(5):H2006-H2017; WO 2016/131137; WO 2018/098597; and U.S. Pat. No. 9,453,201.

Multipotent cells such as human mesodermal cells and cardiac progenitor cells may also be used. As used herein, a “multipotent” cell refers to a cell that is capable of giving rise to more than one cell type upon differentiation. Multipotent cells have more limited differentiation potential than pluripotent cells.

In some other embodiments, the source of cells is differentiated somatic cells that may be reprogrammed into cardiomyocyte cells. For example, the source of cells may be fibroblasts (see, e.g., Engel and Ardehali, Stem Cells Int. (2018) 2018:1-10). Direct reprogramming of fibroblasts into cardiomyocyte-like cells by overexpressing the cardiac developmental transcription factors Gata4, Mef2c, and Tbx5 (GMT) has been reported (Ieda et al., Cell. (2010) 142(3):375-86). In some embodiments, AVNLPCs may then be enriched from a population of cardiomyocyte cells using methods described herein.

Developmentally, cardiac progenitor cells, also referred to interchangeably herein as cardiomyocyte progenitor cells or cardiovascular progenitor cells, are derived from cardiac mesodermal cells, that will upregulate the expression of cardiomyocyte markers cTNT and NKX2-5 between, for example, days 6-20 of differentiation. Cardiac progenitor cells are post-mesoderm phase cells and include cells that do not yet express detectable levels of the cardiomyocyte marker cTNT or NKX2-5 (e.g., day 4 and day 5 cells) as well as cells that do express cTNT or NKX2-5 (e.g., day 6 to day 20 cells). For example, cardiac progenitors will express cTNT at about 12 days to about 20 days of differentiation. As opposed to, e.g., cardiomyocytes into which cardiac progenitor cells can differentiate into, cardiac progenitor cells are not contractile. Cardiac progenitor cells can be subclassified as early stage cardiac progenitor cells which do not express detectable levels of NKX2-5 (i.e., they are NKX2-5⁻) or express NKX2-5 and do not express cTNT (e.g., days 4 to 7) and late stage cardiac progenitor cells which express detectable levels of NKX2-5 (i.e., they are NKX2-5⁺) optionally in combination with cTNT (e.g., days 8 to 20).

One method for generating human cardiac progenitors from human pluripotent stem cells (hPSCs; e.g., hESCs and human iPSCs) involves (i) inducing hPSCS to differentiate into mesoderm by contacting the PSCs with a medium comprising an activator of the activin signaling pathway (e.g., an activin) and an activator of a bone morphogenetic protein 4 (BMP4) receptor (e.g., BMP4); and (ii) inducing the mesoderm to differentiate into cardiac progenitors by contacting the mesodermal cells with a Wnt signaling antagonist.

In some embodiments, the human pluripotent stem cells used to generate hPSCs are not derived from a human embryo or are not human embryonic stem cells.

In other embodiments, the hPSCs are generated from human pluripotent stem cells without involving destruction of a human embryo.

Activins are members of the transforming growth factor beta (TGF-β) family of proteins produced by many cell types throughout development. Activin A is a disulfide-linked homodimer (two beta-A chains) that binds to heteromeric complexes of a type I (Act RI-A and Act RI-B) and a type II (Act RII-A and Act RII-B) serine-threonine kinase receptor. Activins primarily signal through SMAD2/3 proteins when the activated activin receptor complex phosphorylates the receptor-associated SMAD. The resulting SMAD complex regulates a variety of functions, including cell proliferation and differentiation.

Bone morphogenic proteins (BMPs) are part of the transforming growth factor beta superfamily. BMPs bind to two different types of serine-threonine kinase receptors known as BMPR1 (also referred to as BMP type I receptors) and BMPR2 (also referred to as BMP type II receptors). Signal transduction via these receptors occurs via SMAD and MAP kinase pathways to effect transcription of BMP4's target genes. Various BMPs are suitable for use in generating the cells provided herein, including BMP4, BMP2 and the small molecule BMP4 agonist SB 4 (Tocris Bioscience). As used herein, the term “BMP component” refers to activators of BMP signaling which bind to BMPR1 and BMPR2 receptors and mediate BMP signaling activation by phosphorylation of SMAD1, SMAD4, SMAD5 and/or SMAD8, for example BMP2, BMP 4 and SB 4.

Wnt signaling antagonists are molecules (e.g., a chemical compound; a nucleic acid, e.g., a non-coding RNA; a polypeptide; and a nucleic acid encoding a polypeptide) that antagonize the Wnt signaling pathway, thus resulting in decreased pathway output (i.e., decreased target gene expression). For example, a Wnt signaling antagonist can function by destabilizing, decreasing the expression of, or inhibiting the function of a positive regulatory component of the pathway, or by stabilizing, enhancing the expression or function of a negative regulatory component of the pathway. Thus, a Wnt signaling antagonist can be a nucleic acid encoding one or more negative regulatory components of the pathway. A Wnt signaling antagonist can also be a small molecule or nucleic acid that stabilizes a negative regulatory component of the pathway at either the mRNA or the protein level. Likewise, a subject Wnt signaling antagonist can be a small molecule or nucleic acid inhibitor (e.g., microRNA, shRNA, etc.) of a positive regulatory component of the pathway that inhibits the component at the mRNA or protein level. In some embodiments, the Wnt signaling antagonist is a small molecule chemical compound (e.g., Xav-939, C59, ICG-001, IWR1, IWP2, IWP4, IWP-L6, pyrvinium, PKF115-584, and the like). In particular embodiments, Wnt antagonism may be achieved by the combined use of Xav-939 and C59 or the combined use of Xav-939 and IWP-L6. See also US20180251734.

Wnt signaling agonists include, for example, any molecule (e.g., a protein, a chemical compound; a nucleic acid, e.g., a non-coding RNA: a polypeptide; and a nucleic acid encoding a polypeptide) that agonizes the Wnt signaling pathway via stabilization of β-catenin and thereby increasing nuclear β-catenin levels, for example, WNT3a, CHIR-99021, TWS119, BIO, SB 216763, SB 415286, CHIR-98014, synthetic Frizzled and LRP5/6 agonists (PMID: 31452509).

For example, to generate cardiac progenitor cells, the PSCs may first be induced to aggregate to form embryoid bodies (EBs), which are three-dimensional (3D) aggregates of pluripotent stem cells capable of differentiating into the three embryonic germ layers (endoderm, ectoderm and mesoderm). To do so, the PSCs (e.g., hPSCs) may be cultured in an EB medium comprising a BMP component (e.g., BMP4), optionally further comprising a Rho-associated protein kinase (ROCK) inhibitor, for a period of time (e.g., 8-24 h) to generate embryoid bodies. The EB medium may be made with a Roswell Park Memorial Institute (RPMI) base medium (optionally with B27 supplement), a Dulbecco's Modified Eagle Medium (DMEM) base medium, an Iscove's Modified Dulbecco's Media (IMDM) base medium, or StemPro®-34, with the BMP component and/or ROCK inhibitor added to it. In some embodiments, the concentration of BMP4 in the EB medium is between about 0.1 and 10 ng/ml (e.g., about 0.5-5 ng/ml, or about 0.1, 0.5, 1, 2, 3, 4, 5, 6, 7, 8, 9, or 10 ng/ml). In some embodiments, a ROCK inhibitor (e.g., Y-27632; Biotechne-Tocris #1254) in the EB medium may range in 1-20 μM (e.g., 5-15 μM such as 10 μM). For example, the EB medium may contain 1 ng/mL BMP4 and 10 μM Y-27632.

The EBs may then be cultured in a first differentiation medium (mesoderm induction medium) comprising activin A, BMP4, and optionally fibroblast growth factor-basic (bFGF; also known as basic FGF, FGF-basic, FGF-beta, FGF2, heparin binding growth factor, or FGF family members bind heparin). The mesoderm induction medium may be made with an RPMI base medium (optionally with B27 supplement), a DMEM base medium, an IMDM base medium, or StemPro®-34, with the indicated factors added to it. The selection of Activin A, BMP4, and bFGF concentrations may be based on identification of a mesoderm population that contains a low proportion of ALDH+ cells and a low proportion of CD235a+ cells and generates a high proportion of cTNT+ cells at day 20. In some embodiments, the concentration of BMP4 in the mesoderm induction medium is between about 0.1 and 100 ng/ml (e.g., about 1-10 or 1-15 ng/ml; or about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 ng/ml). In some embodiments, the concentration of BMP4 in the mesoderm induction medium is between about 1-10 ng/ml (e.g., 3-8 or 3-5 ng/ml). In some embodiments, the concentration of BMP4 in the mesoderm induction medium is about 2.0, 2.5, 3.0, 3.5, 4.0, 4.5, 5.0, 5.5, 6.0, 6.5, or 7.0 ng/ml. In some embodiments, the mesoderm induction medium includes Activin A at a concentration of about 0.1 and 100 ng/ml (e.g., about 1-10 or 1-15 ng/ml; or about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 ng/mL). In some embodiments, the concentration of Activin A in the mesoderm induction medium is between about 1-15 ng/ml (e.g., 2-12, 1-5, or 2-4 ng/ml). In some embodiments, the concentration of Activin A in the mesoderm induction medium is about 1.0, 1.5, 2.0, 2.5, 3.0, 3.5, 4.0, 4.5, 5.0, 5.5, or 6.0 ng/ml.

In particular embodiments, the mesoderm induction medium contains about 6 ng/ml BMP4, about 4 ng/mL Activin A, and about 5 ng/mL bFGF. In other embodiments, the mesoderm induction medium contains about 8 ng/mL BMP4, about 12 ng/ml Activin A, bFGF as described herein, and optionally CHIR-99021 as described herein. In some embodiments, the mesoderm induction medium contains about 3 ng/mL BMP4, about 2 ng/ml Activin A, bFGF as described herein, and optionally CHIR-99021 as described herein. In select embodiments, the mesoderm induction medium contains about 5 ng/mL BMP4, about 4 ng/ml Activin A, bFGF as described herein, and optionally CHIR-99021 as described herein. The hPSCs may be cultured in the mesoderm induction medium for about 1-3 days (e.g., 1, 1.5, 2, 2.5, or 3 days).

In some embodiments, the mesoderm induction medium additionally contains 0.1-30 ng/mL bFGF (e.g., about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 ng/mL). The mesoderm induction medium, in some embodiments, contains a Wnt signaling agonist (e.g., 1-10 μM CHIR-99021).

In some embodiments, the mesoderm induction medium includes a ratio of BMP4 concentration to Activin A concentration greater than 1 (e.g., 1, 1.1, 1.2, 1.3, 1.4, 1.5, 1.6, 1.7, 1.8 1.9, 2.0, or greater than 2.0). In other embodiments, the mesoderm induction medium includes a ratio of BMP4 concentration to Activin A concentration less than 1 (e.g., 0.9, 0.8, 0.7, 0.6, 0.5, 0.4, 0.3, 0.2, 0.1, or less than 0.1). In some embodiments, the range of concentration ratios of BMP4 to Activin A is between 1-5 (e.g., 1-5, 1-4, 1-3, 1-2, 1-1.5, 1-1.2, 1-1.1).

After this culturing step, the cells may be further cultured for at least 1-3 days (e.g., 1, 1.5, 2, 2.5, or 3 days) in a second differentiation medium (cardiac induction medium) comprising a Wnt signaling antagonist, such as IWP2, and optionally comprising Vascular Endothelial Growth Factor (VEGF). The cardiac induction medium can be made with an RPMI base medium (optionally with B27 supplement), a DMEM base medium, an IMDM base medium, or StemPro®-34, with the indicated factors added to it. In some embodiments, the cardiac induction medium may contain IWP2 at 0.1-10 μM such as 0.5, 1, 2, 3, 4, 5, 6, 7, 8, 9, or 10 μM, and optionally VEGF at 0.1-30 ng/ml (e.g., 5-10 or 5-15 ng/ml; or 0.5, 1, 2, 3, 4, 5, 6, 7, 8, 9, 10, 11, 12, 13, 14, 15, 16, 17, 18, 19, 20, 25, or 30 ng/ml).

After incubation in the cardiac induction medium, the cells may be further cultured for another one to three weeks (e.g., 7-15 days) in a base cardiac medium to obtain a cell population comprising cardiomyocytes. The base cardiac medium may be, for example, an RPMI base medium (optionally with B27 supplement), a DMEM base medium, an IMDM base medium, or StemPro®-34 that is optionally supplemented with VEGF (e.g., at 0.1-30 ng/ml as listed above).

In some embodiments, the EBs are induced to differentiate into cardiac progenitor cells (and eventually cardiomyocytes) in an EB differentiation media, commonly known as EB20 (see, e.g., Lee et al., Circ Res. (2012) 110(12):1556-63). The cardiac progenitors may then be further cultured in a base cardiac medium, such as an RPMI base medium (optionally with B27 supplement), a DMEM base medium, an IMDM base medium, or StemPro®-34, to obtain a cell population comprising human cardiomyocytes (e.g., human cardiac troponinT (cTnT+ cells). The base cardiac medium may contain VEGF as described above.

An alternative method for generating human cardiac progenitors from hPSCs (e.g., hESCs and human iPSCs) involves (i) activating Wnt/β-catenin signaling in hPSCs to obtain a first cell population; and (ii) inhibiting Wnt/β-catenin signaling in the first cell population to obtain a second cell population comprising cardiomyocyte progenitors. In some embodiments, small molecules may be used to sequentially activate and inhibit Wnt/β-catenin signaling. Activation of Wnt/β-catenin signaling in hPSCs may be achieved by contacting the hPSCs with a Wnt signaling agonist. In some embodiments, a Wnt signaling agonist functions by stabilizing β-catenin, thus allowing nuclear levels of β-catenin to rise. β-catenin can be stabilized in multiple ways. As multiple negative regulatory components of the Wnt signaling pathway function by facilitating the degradation of β-catenin, a subject Wnt signaling agonist can be a small molecule or nucleic acid inhibitor (e.g., microRNA, shRNA, etc.) of a negative regulatory component of the pathway that inhibits the component at the mRNA or protein level. For example, the Wnt signaling agonist is an inhibitor of glycogen synthase kinase-3β (GSK-3β). In some such embodiments, the inhibitor of GSK-3β is a small molecule chemical compound (e.g., CHIR-99021, TWS119, BIO, SB 216763, SB 415286, CHIR-98014, and the like). Inhibition of Wnt/β-catenin signaling may be achieved by contacting the cells that were previously contacted with the Wnt signaling agonist, with a Wnt signaling antagonist, such as those described above. In general, after ending the inhibition of Wnt/β-catenin signaling, cardiac progenitors may be further cultured in a base cardiac medium, such as an RPMI base medium (optionally with B27 supplement), a DMEM base medium, an IMDM base medium, or StemPro®-34, to obtain a cell population comprising human cardiomyocytes (e.g., human cardiac troponinT (cTnT+ cells).

In an exemplary, nonlimiting protocol based on the protocol described in Lian et al., supra, cardiomyocytes may be generated from human PSCs via cardiac induction using CHIR as follows. At day −1, 6E6 hPSCs may be plated and cultured on Vitronectin-coated six-well plates in E8 medium and allowed to attach to the plates overnight. At day 0, cell culture medium may be prepared by adding CHIR-99021 (“CHIR”; Tocris 4423/10) to basal cardiomyocyte (CM) medium (RPMI (with L-Glutamine)/B-27 without insulin, plus 213 μg/mL L-ascorbic acid 2-phosphate (Sigma)) to reach a CHIR concentration of 2, 4, 6, 8, 10, or 12 μM. The old medium in the plates may be replaced with 4 ml per well of CHIR-supplemented basal CM medium. Optimization of CHIR concentration may be desirable (e.g., a range of 2-12 μM CHIR may be tested). At day 1, the culture medium may be removed by aspiration. The wells may be washed once with DMEM to remove debris. Then room-temperature RPMI/B-27/without insulin medium may be added at a volume of 4 ml per well. The plates may be incubated at 37° C., 5% CO2. At days 2 to 3, the culture medium may be removed by aspiration. The wells may be washed once with DMEM to remove debris. Then IWR1 may be added to 4ml of fresh RPMI/B-27/without insulin medium, to reach a final IWR1 concentration of 2.5 M. At day 5, the culture medium may be replaced with room-temperature RPMI/B-27/without insulin medium at a volume of 4 ml per well. The plates may be incubated at 37°° C., 5% CO2. At days 5, 6, 7, the cell culture comprises cardiac progenitor cells. From day 7 and on, the culture medium may be replaced with room-temperature RPMI/B-27 medium at a volume of 4 ml per well to generate cardiomyocytes. The plates may be incubated at 37° C., 5% CO2. Cardiomyocytes may be counted by flow cytometry (cTNT/NKX2-5). Robust spontaneous contraction should occur by day 12. The cells can be maintained with this spontaneously beating phenotype for more than 6 months. This protocol may be scaled up to produce large quantities of cardiac progenitors and/or cardiomyocytes. For example, bioreactors, large roller bottles, and other culturing devices may be used in lieu of multi-well tissue culture plates.

The starting population of cardiac progenitor cells can be provided as a mixed population, e.g., comprising cells that have differentiated into cardiomyocytes.

In some embodiments, the starting population is cardiovascular mesoderm cells. Such cells, express surface markers PDGFR-alpha (high) and KDR (low) (U.S. Pat. No. 10,561,687). In addition, these cells express surface marker CD56 and express MESPI and T (Brachyury) by Q-RT-PCR, and can give rise to cTNT+ cardiomyocytes.

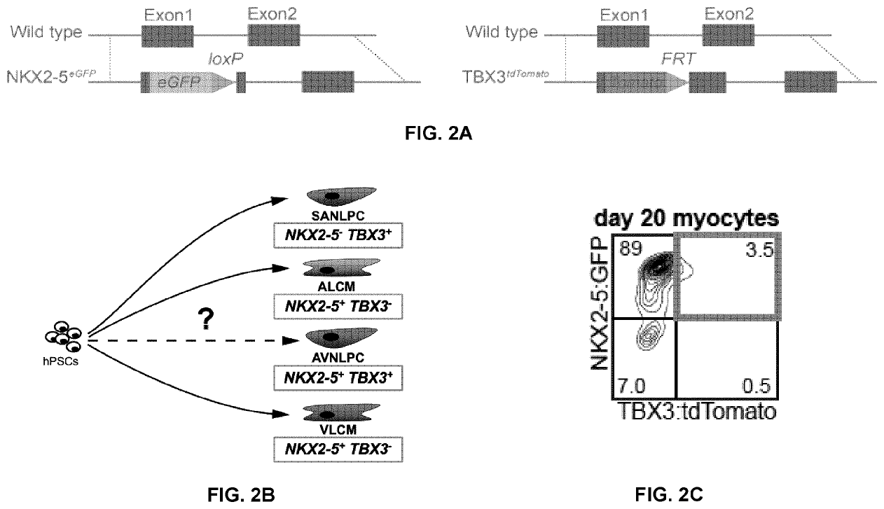

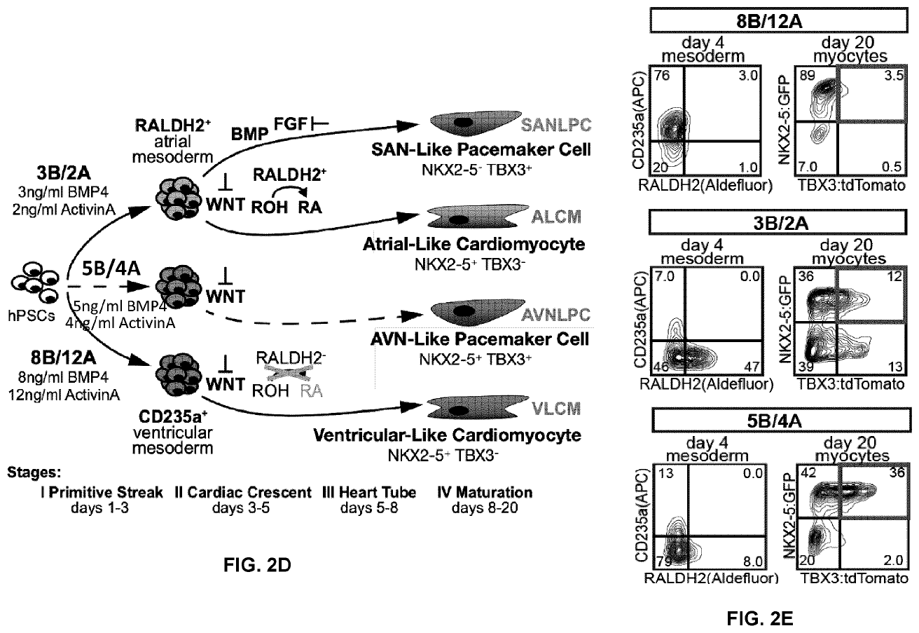

In particular embodiments, the culture methods comprise: a) incubating the hPSCs in an embryoid body medium comprising a BMP component (e.g., BMP4), optionally further comprising a Rho-associated protein kinase (ROCK) inhibitor, for a period of time to generate embryoid bodies; b) incubating the embryoid bodies in a mesoderm induction medium comprising a BMP component (e.g., BMP4), and an activin component (e.g., activin A), and optionally a FGF component (e.g., bFGF), for a period of time to generate cardiovascular mesoderm cells; c) incubating the cardiovascular mesoderm cells in a cardiac induction medium comprising a Wnt inhibitor, optionally IWP2, and VEGF for a period of time to generate cardiovascular progenitor cells, and (d) incubating the cardiovascular progenitor cells in a basic medium comprising VEGF for a period of time to generate a population of cardiomyocytes enriched for AVNLPCs. As used herein, the term “activin/Nodal component” refers to activators of the Activin/Nodal growth factor signaling pathway which bind to serine-threonine kinase receptors known as type I (Act RI-A and Act RI-B) and type II (Act RII-A and Act RII-B) and mediate Activin/Nodal pathway signaling by phosphorylation of SMAD2, SMAD3 and/or SMAD4, for example Activin A and Nodal. Cardiomyocytes in the culture can be isolated by using a cardiomyocyte-specific surface marker such as SIRPA and thymocyte differentiation antigen 1 (THY-1/CD90) optionally wherein the isolated population is SIRPA+CD90−. See also FIG. 2A.

The medium used at the various differentiation stages as described above can be made with any suitable base medium, which includes, without limitation, an RPMI base medium (optionally with B27 supplement), a DMEM base medium, an IMDM base medium, or StemPro®-34, with the indicated factors (e.g., cytokines and small molecules) added to the base medium.

A non-limiting, exemplary method is described as follows, in which the starting cell population are human embryonic stem cells and human induced pluripotent stem cells (collectively “human pluripotent stem cells” or hPSCs) and are differentiated into AVNLPCs in a stepwise fashion. In the first step, the hPSC starting cells are cultured in a culture medium containing a bone morphogenetic protein component (e.g., BMP2 or BMP4) and a Rho-associated protein kinase (ROCK) inhibitor to generate embryoid bodies (day 0 of the differentiation protocol).

In the second step, the embryoid bodies are induced to become a cardiogenic mesoderm by incubation with a BMP (e.g., BMP4 at 1-100 ng/mL), an Activin/Nodal activator (e.g., Activin A at 1-100 ng/ml), FGF (e.g., bFGF at 1-20 ng/mL) and a Wnt signaling agonist (e.g., CHIR-99021 at 1-10 μM) (day 1 of the differentiation protocol). In some embodiments, this cardiogenic mesoderm is analyzed at day 3/4 of differentiation for the expression of PDGFR alpha, RALDH2, and CD235a; cardiogenic mesoderm cells with high potential to develop into AVNLPCs are characterized by PDGFRa⁺RALDH2⁻CD235a^{low}.

In the third step, the cardiogenic mesoderm is induced to generate cardiac progenitors using a Wnt antagonist (e.g., IWP2 at 0.5-10 μM) together with VEGF activation (e.g., in the presence of VEGF at 1-20 ng/mL) (day3 of the differentiation protocol).

In the fourth step, the cardiac progenitor cells are induced to acquire an AVN phenotype by incubation with a Wnt agonist (e.g., CHIR-99021 at 1-20 μM), optionally in the presence of VEGF (5 ng/mL) (between days 8-15, for example between days 8 to 11). In preferred embodiments, the incubation is carried out with a Wnt agonist (e.g., CHIR-99021 optionally at 1-20 μM or any subrange therein) in late stage cardiac progenitors which exhibit detectable levels of NKX2-5 expression (e.g., are NKX2-5⁺), which can coincide with, for example, day 8 of the differentiation protocol as herein described. In other embodiments, incubation with the Wnt agonist (e.g., CHIR-99021 optionally at 1-20 μM or any subrange therein) is carried out over about 2 to 10 days, or any subrange therein, optionally 4 to 5 days starting on about day 8, for example day 8 to day 11, day 11 to day 15 or day 18 to day 21 or day 8 to day 21 or any subrange therein of the differentiation protocol as herein described. Alternatively, AVN phenotype can be induced by using BMP activation (e.g., in the presence of BMP2 at 10-500 ng/ml), optionally in the presence of VEGF (5 ng/mL) (between days 5-11). Alternatively, AVN phenotype can be induced by incubation of early stage cardiac progenitors (e.g., NKX2-5⁻) with a BMP component (e.g., in the presence of BMP2 at 10-500 ng/ml), optionally in the presence of VEGF (5 ng/ml), for example between days 5-8 of the differentiation protocol as herein described, and incubation of the progenitors at a late stage (e.g., NKX2-5⁺) with a Wnt agonist (e.g., CHIR-99021 at 1-20 μM) for about 4 to 5 days starting on about day 8 to day 11 or any subrange therein, day 11 to day 15 or any subrange therein, or day 18 to day 21 or any subrange therein of the differentiation protocol as herein described. In some embodiments, some overlap between incubation with the BMP component and incubation with the Wnt agonist may occur, for example for about 1 day or on day 8, for about 2 days or on about days 8 to 9 or any subrange therein, for about 3 days or on about days 8 to 10 or any subrange therein or more of the differentiation protocol as herein described, wherein the progenitors are incubated in a medium comprising both a BMP component and a Wnt agonist simultaneously. In embodiments wherein both a Wnt agonist and a BMP component are used, culturing with the BMP component, can for example precede or overlap with culturing with the Wnt agonist.

In the fifth step, the cells continue to be cultured in the presence of VEGF (e.g., 1-10 ng/mL, such as 5 ng/mL) until day 12 to 24 of differentiation.

In some embodiments, AVNLPCs may be identified and further enriched based on the expression of NKX2-5 and TBX3. In addition, AVNLPCs are cardiomyocytes that express the surface marker SIRPA and lack the expression of the non-cardiomyocyte marker CD90. Thus, AVNLPCs can be also enriched and isolated from non-cardiomyocytes by sorting for SIRPA⁺CD90⁻ cardiomyocytes.

Each of these steps are modular and can be modified. In some embodiments, steps 2 and 4 each can result in about 30-50% AVNLPCs. The final cell population may contain 90% or more AVNLPCs.

### II. Pharmaceutical Compositions and Use

The enriched AVNLPC cell preparations of the present disclosure can be used in biological pacemaker therapies to treat a subject (e.g., a human subject) with age-related loss of SAN or AVN function, a congenital disease, or a heart surgery that results in a slow, irregular heartbeat (bradyarrhythmia) with symptoms ranging from fatigue to syncope. In some embodiments, a patient in need of the present cell therapy has bradycardia, arrhythmia, AV-block, i.e., partial AV-conduction block (first and second degree AV-block) as well as complete AV-conduction block (third degree AV-block), left and/or right bundle branch block, atrial fibrillation, or a congenital condition such as Sick Sinus Syndrome and congenital AV-block. In some embodiments, the subject is suffering from one or more previous myocardial infarctions or heart failure. In some embodiments, the type of heart failure is a left-sided heart failure, a right-sided heart failure, a diastolic heart failure, or a systolic heart failure. In some embodiments, the heart failure is congestive heart failure. In some embodiments, patients that are indicated for pacemaker treatment include heart failure patients with reduced left ventricular ejection fraction (e.g., ≤35%), left bundle branch block (Arnold et al., J Am Coll Cardiol. (2018) 72:3112-22), unsynchronized contraction of left and right ventricles (cardiac dyssynchrony; Cleland et al., N Engl J Med. (2005) 15:1539-49), and/or QRS prolongation (Bristow et al., N Engl J Med. (2004) 21:2140-50), and patients with heart failure caused by ischemic cardiomyopathies (myocardial infarction; Bristow, supra).

The AVNLPC cell preparations of the present disclosure may be administered via minimally invasive methods and/or transplanted locally into a subject in need thereof. Various methods are known in the art for administering cells into a patient's heart (e.g., atrium), and include, without limitation, intracoronary administration, intramyocardial administration, or transendocardial administration. AVNLPCs can be introduced to the heart by using a catheter-based approach. The catheter may be inserted via the femoral, subclavian, jugular or axillary vein, or by endocardial transplantation into the ventricle, atrium or AVN region. The cells also can be transplanted into the ventricle, atrium, or AVN region by an epicardial approach, using a needle inserted through the chest. Fluoroscopy (X-ray based method) or 3D mapping can be used to guide the catheter/needle to the intended injection site.

The AVNLPC-enriched cell preparations described herein may be provided in a pharmaceutical composition containing the cells and a pharmaceutically acceptable carrier. In some embodiments, the pharmaceutical composition comprises a population of PSC-derived (e.g., iPSC-derived) AVNLPCs and a pharmaceutically acceptable carrier and/or additives. For example, sterilized water, physiological saline, general buffers (e.g., phosphoric acid, citric acid, and other organic acids), stabilizers, salts, anti-oxidants, surfactants, suspension agents, isotonic agents, cell culture medium that optionally does not contain any animal-derived component, and/or preservatives may be included in the pharmaceutical composition. In some embodiments, a pharmaceutically acceptable carrier can be a hydrogel, for example a collagen or gelatin hydrogel, optionally an extracellular matrix (ECM)-derived hydrogel, optionally formulated for injection. In some embodiments, the pharmaceutical composition is formulated into a dosage form suitable for administration to a subject in need of treatment. In some embodiments, the pharmaceutical composition is formulated into a dosage form suitable for intramyocardial administration, transendocardial administration, or intracoronary administration. For storage and transportation, the cells optionally may be cryopreserved. Prior to use, the cells may be thawed and diluted in a sterile carrier that is supportive of the cell type of interest.

A therapeutically effective number of AVNLPCs are administered to the patient. As used herein, the term “therapeutically effective” refers to a number of cells or amount of pharmaceutical composition that is sufficient, when administered to a human subject suffering from or susceptible to a disease, disorder, and/or condition, to treat, prevent, and/or delay the onset or progression of the symptom(s) of the disease, disorder, and/or condition. It will be appreciated by those of ordinary skill in the art that a therapeutically effective amount is typically administered via a dosing regimen comprising at least one-unit dose.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Further, unless otherwise required by context, singular terms shall include pluralities and plural terms shall include the singular. Throughout this specification and embodiments, the words “have” and “comprise,” or variations such as “has,” “having,” “comprises,” or “comprising.” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

As used in this specification and appended claims, the singular forms “a”, “an” and

“the” include plural references unless the content clearly dictates otherwise. For example, “a pharmaceutically acceptable carrier” includes two or more pharmaceutically acceptable carriers.

As used in this specification and appended claims, the term “or” is generally employed in the sense including “and/or” unless the content clearly dictates otherwise.

The recitation of numerical ranges by endpoints herein includes all numbers and fractions subsumed within that range. For example, “1-10 days” includes, 1, 2, 3, 4, 5, 6, 7, 8, 9, and 10 days. It is to be understood that all numbers and fractions thereof are presumed to be modified by the term “about”.

All terms as used herein are to be understood according to the normal usage in the art.

The definitions and embodiments described in particular sections are intended to be applicable to other embodiments herein described for which they are suitable as would be understood by a person skilled in the art. For example, in the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

According to the present disclosure, back-references in the dependent claims are meant as short-hand writing for a direct and unambiguous disclosure of each and every combination of claims that is indicated by the back-reference.

In order that this invention may be better understood, the following examples are set forth. Any methods and materials similar or equivalent to those described herein can also be used in the practice or testing of the present disclosure. These examples are for purposes of illustration only and are not to be construed as limiting the scope of the invention in any manner.

### EXAMPLES

### Example 1: Differentiation of AVNLPC Cardiomyocytes in Cell Cultures

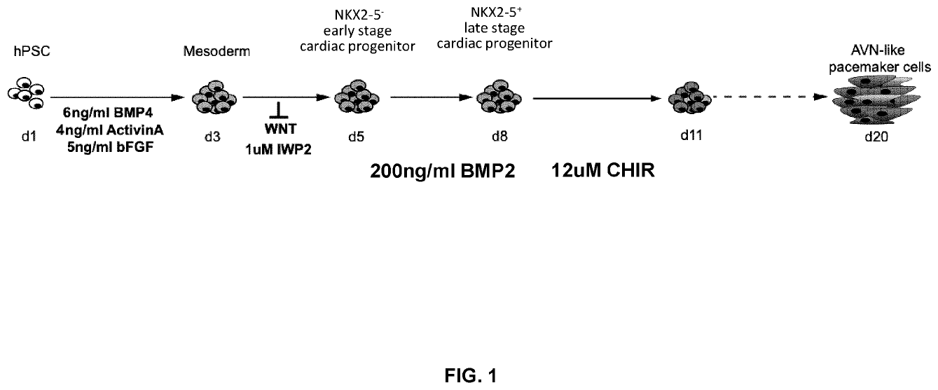

This example describes protocols for differentiating AVNLPCs from human pluripotent stem cells (hPSCs) or human embryonic stem cells (hESCs) (FIG. 1).

First, hPSCs were incubated in media containing BMP and Rho-associated protein kinase (ROCK) inhibitor on an orbital shaker to generate embryoid bodies (differentiation protocol day: 0). Then, cardiogenic mesoderm was induced using media containing 1-100 ng/mL BMP4, 1-100 ng/ml Activin A, 1-20 ng/mL bFGF, and 1-10 μM CHIR-99021 (differentiation protocol day: 1). Compared with media containing 8 ng/ml BMP4 and 12 ng/ml Activin A (i.e., “8B/12A”) or 3 ng/ml BMP4 and 2 ng/ml Activin A (i.e., “3B/2A”), media containing 5 ng/ml BMP4 and 4 ng/ml Activin A (i.e., “5B/4A”) induced cardiogenic mesoderm that produced a higher proportion of AVNLPCs during maturation (FIGS. 2D-E). The cardiogenic mesoderm can be analyzed at day 3/4 of differentiation for the expression of PDGFRa, RALDH2 and CD235a, and should be PDGFRa+RALDH2− and CD235a^{low} (max 50% positive). Cardiogenic mesoderm produced using 5B/4A media, compared with 8B/12A or 3B/2A media, contained a higher proportion of RALDH2− and CD235a^{low} cells (FIG. 2E).

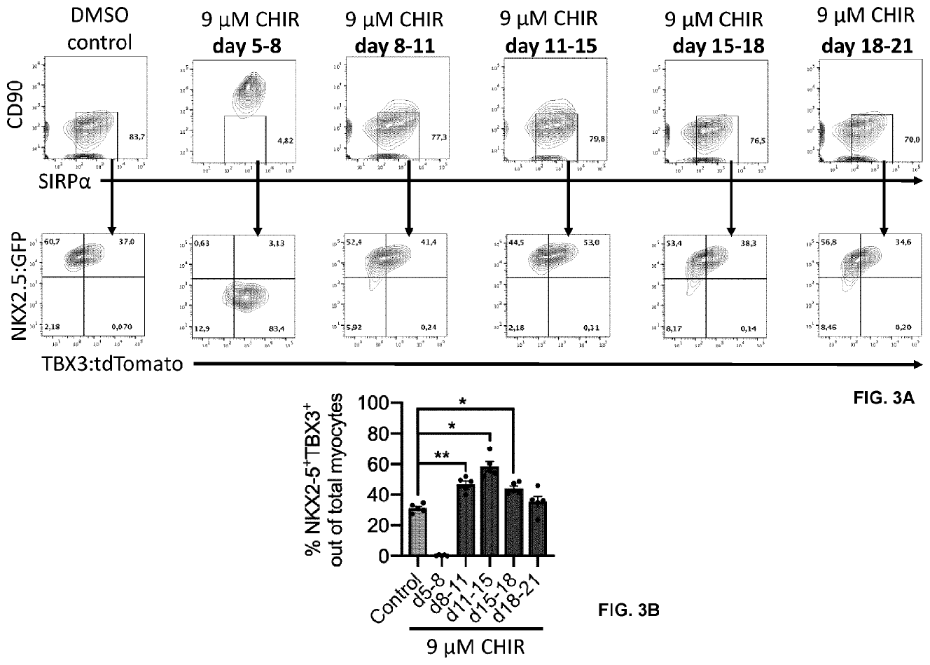

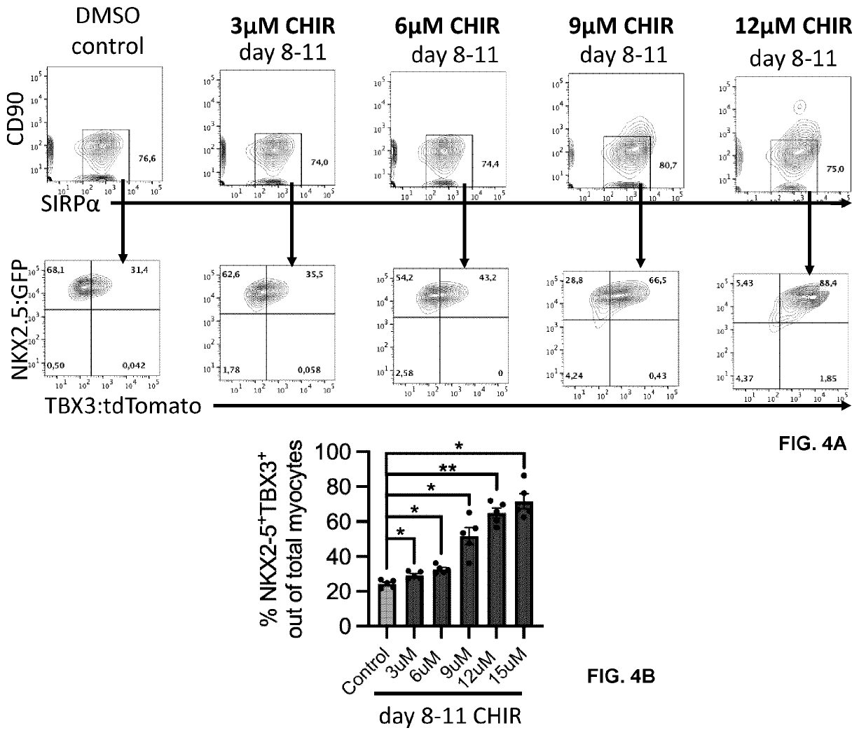

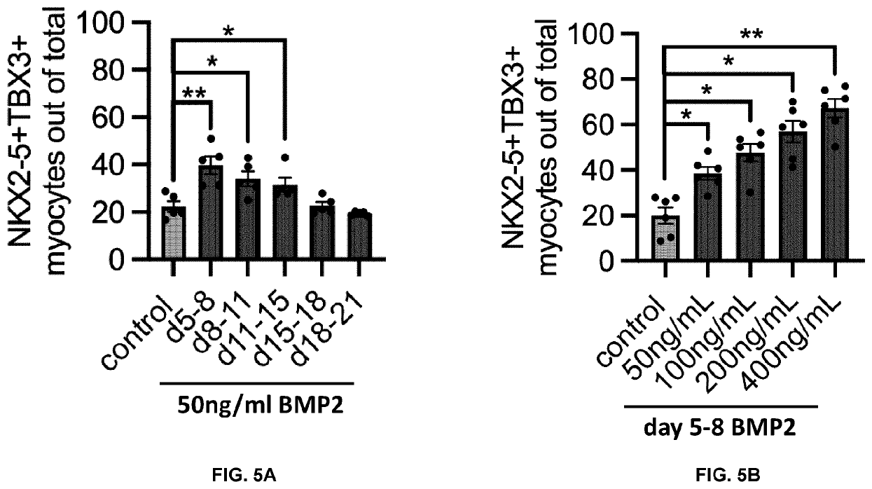

Next, cardiac progenitors were induced using media that contained 0.5-10 μM IWP2 and 1-20 ng/mL VEGF (differentiation protocol day: 3). At this stage, the cardiac progenitors may be a mixed population of early stage (NKX2-5⁻) and late stage (NKX2-5⁺) cardiac progenitors which may be contractile or not. The cardiac progenitors were induced into an AVN phenotype between days 8-18, which yielded relatively more NKX2³⁰ -5⁺TBX3⁺ myocytes when carried out between days 8-11 (FIG. 3A,B), using media that contained 3-15 μM CHIR-99021 and optionally contained 5 ng/mL VEGF (FIG. 4A,B). Relatively more NKX2-5⁺TBX3⁺ myocytes were induced when CHIR-99021 was used at 12-15 μM. Alternatively, the AVN phenotype was induced between days 5-15 (FIG. 5A) using media that contained 50-400 ng/mL BMP2 and optionally contained 5 ng/mL VEGF (FIG. 5B). Relatively more NKX2-5⁺TBX3⁺ myocytes were induced when BMP2 was applied between days 5-8 and when used at 200-400 ng/mL. Culture was continued in media that contained 5 ng/mL VEGF until day 12 of the differentiation protocol. The culture was maintained until day 20 of the differentiation protocol before analysis. Treatment with 15 μM CHIR-99021 from day 8-11 results in 72=10% NKX2-5⁺ TBX3⁺myocytes of total cells present in culture at day 20. Treatment with 400 ng/ml BMP2 from day 5-8 results in 67±10% NKX2-5³⁰ TBX3 myocytes of total cells present in culture at day 20. FIGS. 3B, 4B, 5A and 5B present the percentage of NKX2-5⁺ TBX3⁺ myocytes of total cells present in the culture.

### Example 2: Identification and Isolation of AVNLPCs in Differentiation Cultures

AVNLPCs are characterized by the expression of the following markers: NKX2-5+, TBX3+, HNC4+, TBX2+, MSX2+, BMP2+and SHOX2−, NPPA−, GJA1−, GJA5− compared to other hPSC-derived cell types such as ventricular (VLCM) or atrial like cardiomyocytes (ALCM), or Sinoatrial node like pacemaker cells (SANLPCs).

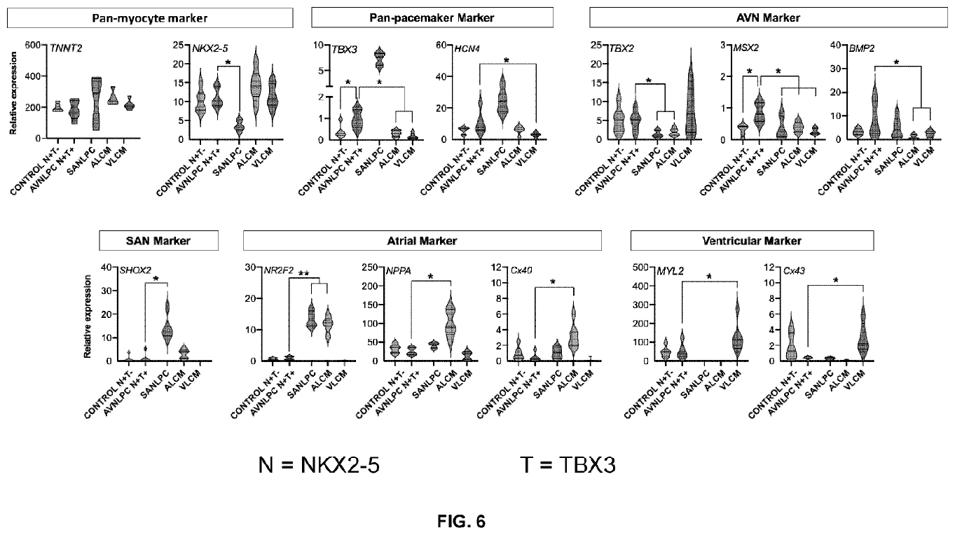

A NKX2-5:GFP+TBX3:tdTomato+ transgenic double reporter hPSC line was cultured using the protocol described in Example 1. AVNLPCs were identified and isolated using Fluorescence-activated cell sorting (FACS), based on their expression of NKX2-5 and TBX3 and the associated expression of the fluorescent reporter (FIG. 2). RT-qPCR was performed using the resulting cell population, which demonstrated that the isolated NKX2-5+TBX3+ cells expressed genes typical of atrioventricular node cells (e.g., TBX2, MSX2, BMP2; FIG. 6).

AVNLPCs express the surface marker SIRPA and lack expression of the non-cardiomyocyte marker CD90. Therefore, AVNLPCs could also be enriched and isolated from non-cardiomyocytes by sorting for SIRPA+ CD90− cardiomyocytes. Alternatively, the PSC-Derived Cardiomyocyte Isolation Kit (Miltenyi) could be used to isolate AVNLPCs.

### Example 3: Functional Testing of AVNLPCs

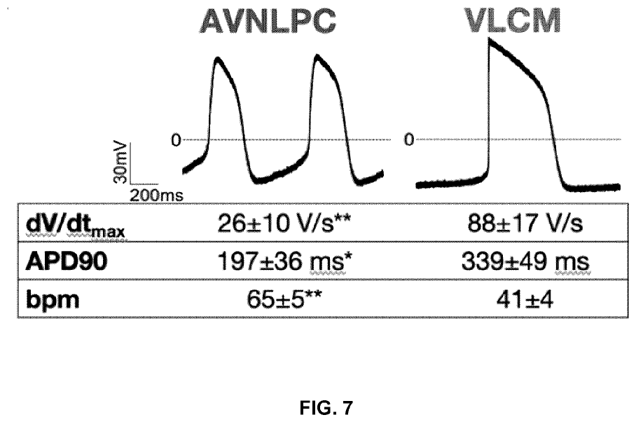

The electrophysiological properties of the NKX2-5+TBX3+ AVNLPCs, identified and isolated as described in Example 2, were determined using single cell patch clamp analysis. The action potentials of NKX2-5+TBX3+ AVNLPCs had properties typical of pacemaker action potentials and differed from the properties of action potentials recorded in NKX2-5+TBX3− ventricular like cardiomyocytes (VLCMs), e.g., AVNLPC action potentials had maximum upstroke velocities of 26±10 V/s and action potential durations at 90% repolarization of 197±36 ms compared to ventricular control cells that had maximum upstroke velocities of 88±10 V/s and action potential durations at 90% repolarization of 339±49 ms (FIG. 7). NKX2-5+TBX3+ AVNLPCs also had a spontaneous beating rate of 65±5 beats per minute, which is comparable to the spontaneous rate of the human AVN.

### Example 4: In Vivo Application of AVNLPCs

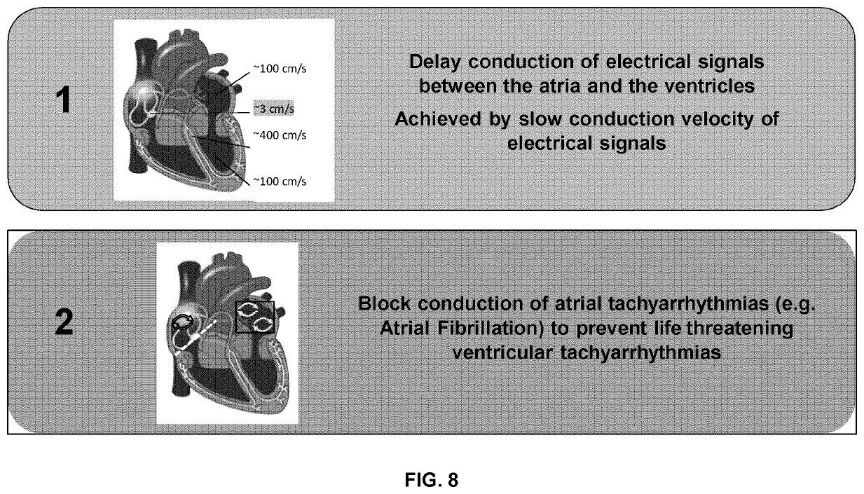

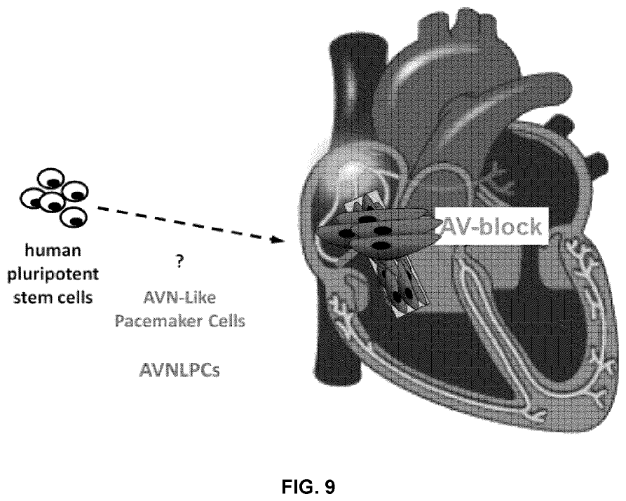

The AVN has specialized conduction properties essential to its function (FIG. 8). AVNLPCs have the potential to be used as a conduction bridge in vivo to treat any type of conduction block, particularly AV-blocks (heart blocks) (FIG. 9).

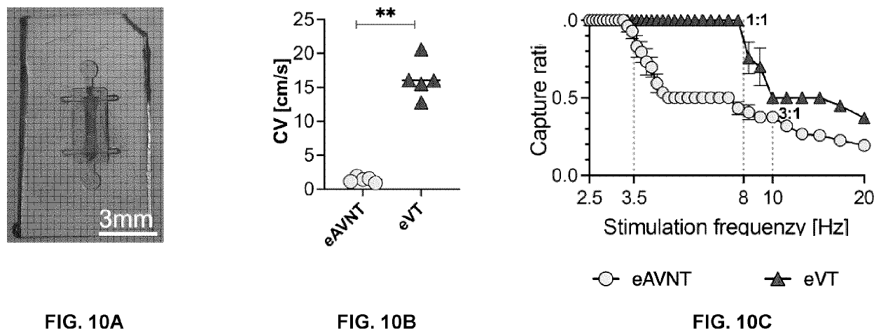

To analyze the conduction properties of AVNLPCs, 3D tissues were generated (fibroblasts and cardiomyocytes 1:3 mix in fibrin gel) containing either AVNLPCs or control VLCMs (FIG. 10A). When embedded into 3D tissues, AVNLPCs show slow conduction velocities of 1.4±0.2 cm/s comparable to the slow conduction via the human AVN (FIG. 10B).

In contrast, VLCM tissues show significantly faster conduction velocities of 16±1 cm/s. Further, AVNLPCs embedded in 3D tissues are able to block the conduction of fast pacing rhythms. They have a maximum capture rate of 3.5 Hz and when paced at 10 Hz only conduct every third beat, resulting in a rate of 3 Hz (FIG. 10C). In contrast, VLCM tissues do not efficiently block the conduction of fast rates and showed a maximum capture rate of 8 Hz. Conduction of a rate of 8 Hz to the ventricular chambers of the heart would be life-threatening. Finally, AVNLPCs also show decremental conduction properties. That is, the faster the tissues are paced, the larger the conduction delay until a block of conduction is reached.

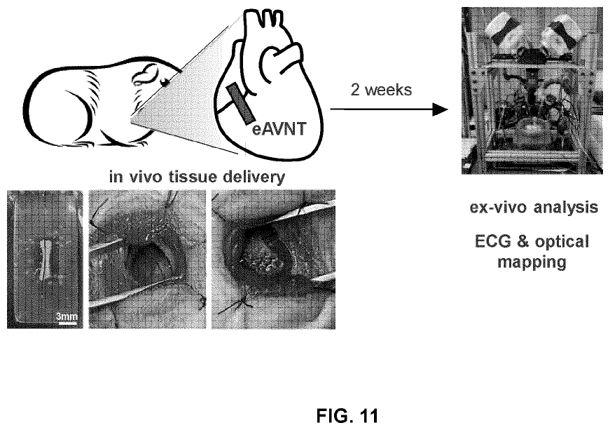

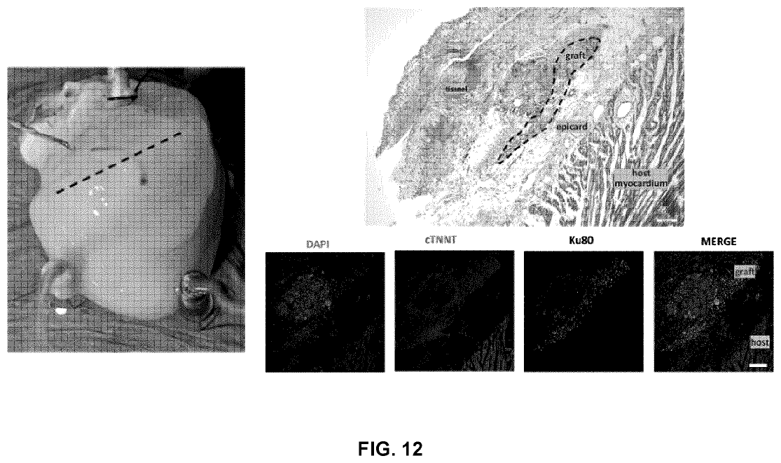

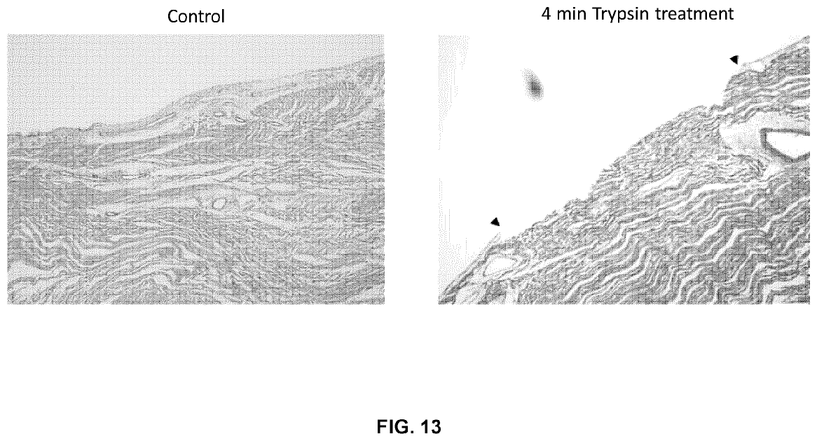

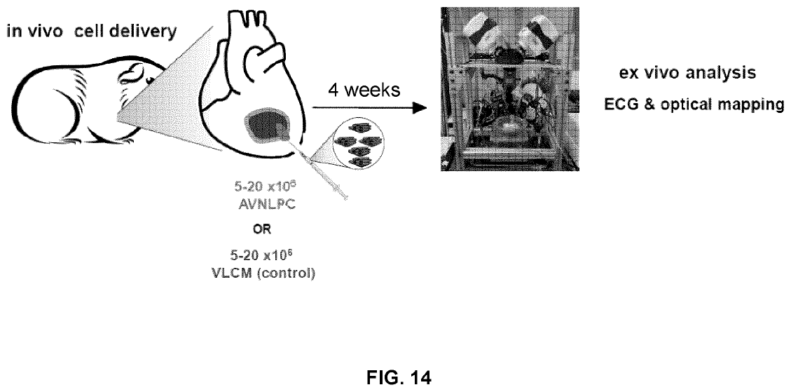

3D tissues containing AVNLPCs can be engrafted on top of the guinea pig heart, such that they span the atria and the ventricle (FIG. 11). To attach the tissues, Tisseel (fibrin glue; Baxter) is used together with SURGICELR (Johnson & Johnson), a degradable cellulose patch. In rare cases (<15%) the tissue graft can electrically integrate with the host heart and conduct electric activity from the atria to the ventricles of the guinea pig heart. In most cases, the AVNLPCs grafts are insulated from the host heart myocardium (FIG. 12). To enhance electrical integration, the epicardial layer of the host heart can be removed using dissociation agents, such as trypsin (FIG. 13), or surgical methods. In another approach, AVNLPCs can be injected into the host heart, such as into the border zone of a scar that is created by cryoablation (FIG. 14). Alternatively, in larger animal models, e.g., pigs or dogs, AVNLPCs can be injected directly into the AVN region near an AVN injury.

### Example 5: Dual Color Optical Mapping to Assess Electrical Integration

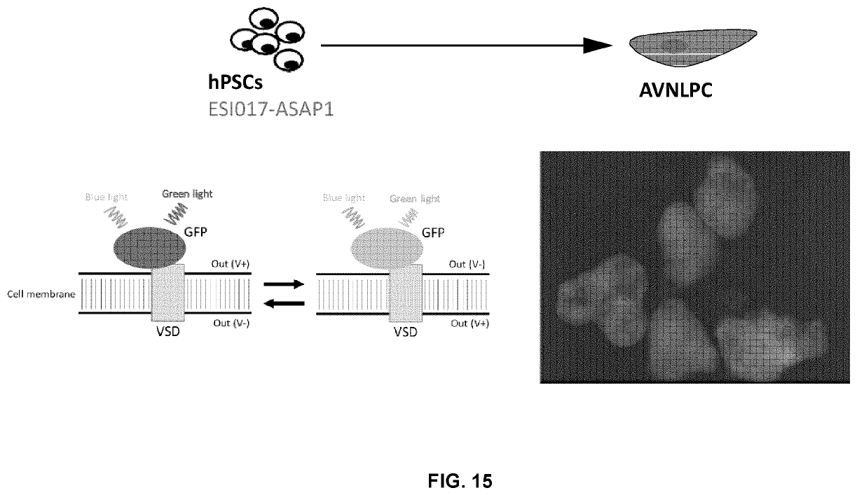

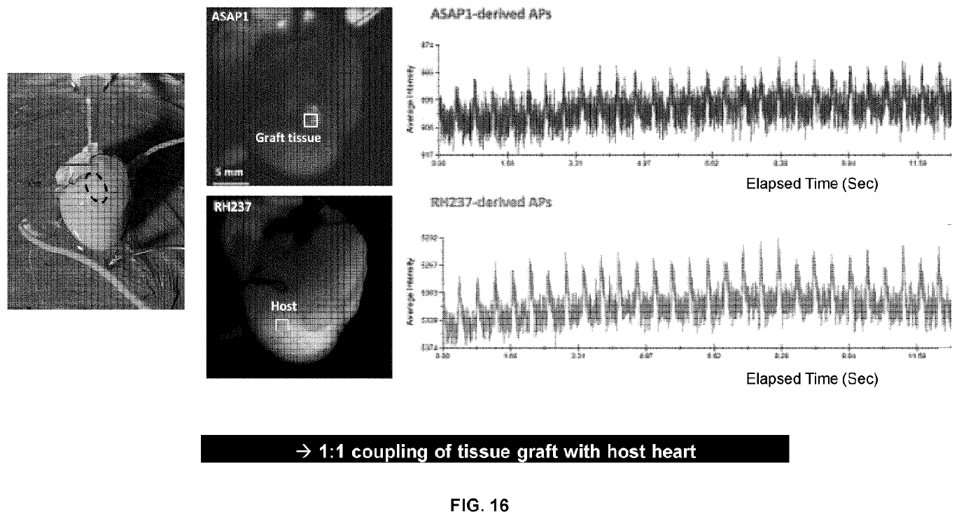

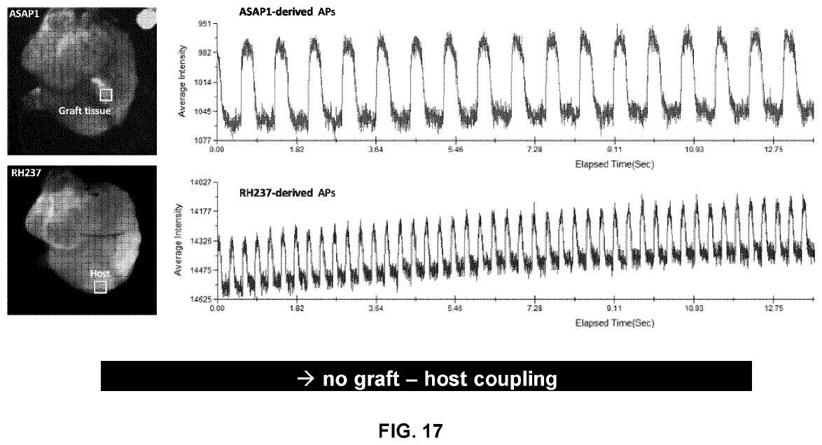

To assess the electrical integration of the graft tissue with the host tissue, dual color optical mapping was used, wherein hPSC-derived cardiomyocytes were generated from a reporter cell line carrying the ASAP1 voltage sensor (FIG. 15). The ASAP1 voltage sensor uses modified GFP fused to a voltage-sensitive domain. The GFP fluorescence intensity changes depending on the voltage change across the cell membrane and decreases intensity during the voltage change associated with an action potential. By generating AVNLPCs using an ASAP1 hPSC line, voltage changes in engrafted engineered tissue were measured, while the voltage changes of the host heart were monitored using the voltage sensitive dye RH237. The changes in fluorescence in both sensors were simultaneously measured using two cameras. AVNLPC graft tissues that did electrically couple with the host heart produced fluorescent changes in both voltage indicators correlated in time (FIG. 16). AVNLPC graft tissues that did not electrically couple with the host heart produced asynchronous fluorescent changes in the voltage indicators (FIG. 17).

When AVNLPCs were directly injected into the border zone of a cryoablation-induced scar, electrical coupling of the engrafted cells with the host heart was enhanced compared to the engraftment of engineered tissues on the surface of the heart. Cells were injected from the healthy host myocardium towards the cryoablation-induced scar tissue to allow for electrical coupling with the host heart on one side of the graft and for measurement of graft conduction properties on the other side of the graft that resides within the scar tissue.

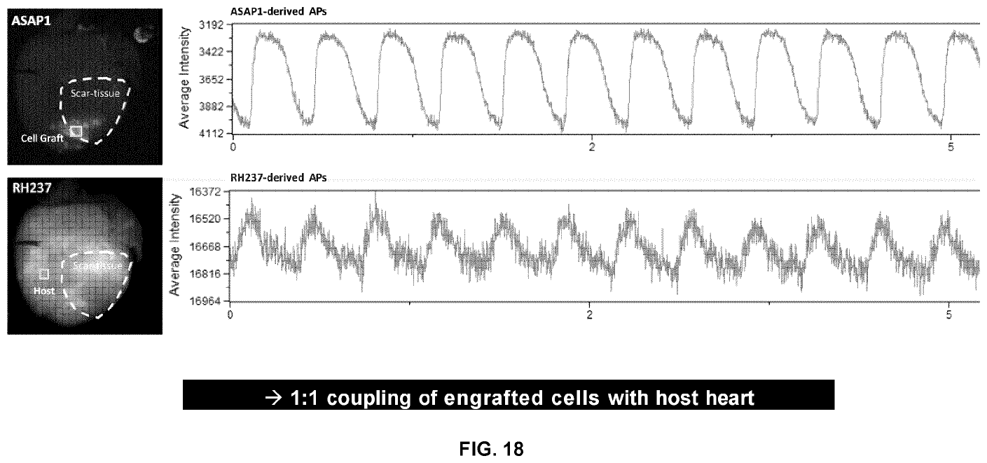

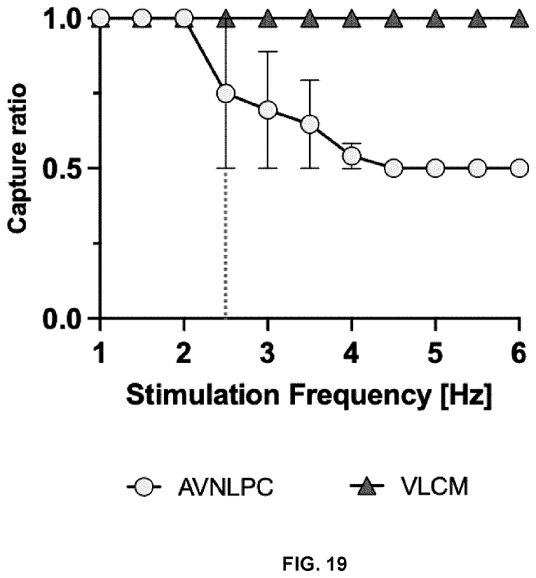

(FIG. 18). To test the ability of the grafts to conduct electric activity from the healthy myocardium into the scar, the host hearts spontaneous rhythm is blocked via methacholine perfusion. This allows to then use and electrode and pace the ventricles with increasing frequencies (1-6 Hz) to assess maximum capture ratios of the grafts. Four weeks after engraftment AVNLPC cell grafts loose 1:1 capture at 2.5 Hz (FIG. 19). When paced at 6 Hz the AVNLPC grafts only conducted every 2nd beat, resulting in a rate of 3 Hz. In contrast VLCM cell grafts maintain 1:1 capture up to 6 Hz. This data indicates that AVNLPCs retain their important AVN-like conduction properties during long term engraftment in vivo.

What is claimed is:

1. A method of generating a population of cardiomyocytes enriched for atrioventricular node-like pacemaker cells (AVNLPCs), comprising:
(a) providing a starting population of human cardiac progenitors; and
(b) culturing the human cardiac progenitors in a medium comprising a Wnt agonist or a bone morphogenetic protein (BMP) component to generate AVNLPCs.

2. The method of claim 1, wherein the culturing comprises culturing the human cardiac progenitors in a medium comprising a bone morphogenetic protein (BMP) component, optionally for about 4 days, and culturing the progenitors in a medium comprising a Wnt agonist, optionally for about 4 to about 5 days.

3. The method of claim 2, wherein the culturing at least partially comprises culturing the human cardiac progenitors in a medium comprising a bone morphogenetic protein (BMP) component and a Wnt agonist, optionally for about 1 day.

4. The method of any one of claims 1-3, wherein the AVNLPCs are characterized by being NKX2-5⁺TBX3⁺.

5. The method of any one of claims 1-4, wherein the medium further comprises VEGF, optionally at 1-20 ng/ml, further optionally at 5 mg/mL.

6. The method of any one of claims 1-5, wherein the Wnt agonist is CHIR-99021, optionally at 1-20 μM or any range between 1-20 μM, optionally 1, 2, 3, 4, 5, 6,7, 8, 9, 10, 11, 12, 13, 14, 15, 16, 17, 18, 19, or 20 μM.

7. The method of claim 6, wherein the medium comprises CHIR99021 and VEGF, optionally wherein the culturing step is performed for about 1-10 days, optionally about 2-10, 2-8, 3-6, 3-5, or 5-10 days, optionally about 3 or 4 days.

8. The method of any one of claims 1-5, wherein the BMP component is BMP2, optionally at 10-500 ng/mL.

9. The method of claim 8, wherein the medium comprises BMP2 and VEGF, optionally wherein the culturing step is performed for about 1-10 days, optionally about 2-10, 2-8, 3-6, 3-5, or 5-10 days, optionally about 3 or 4 days.

10. The method of any one of claims 1-9, wherein the cardiac progenitor cells are obtained by culturing cardiogenic mesoderm cells in the presence of a Wnt inhibitor and VEGF, optionally for about 2 to 7 days.

11. The method of claim 10, wherein the Wnt inhibitor is IWP2, optionally at 0.5 to 10 μM, and/or the VEGF is at 1-20 ng/mL.

12. The method of claim 10 or 11, wherein the cardiogenic mesoderm cells are obtained by culturing embryoid bodies in the presence of a cardiac progenitor inducing medium comprising one or more, or all, of BMP component and an activin/Nodal component, FGF, and a Wnt agonist, optionally for about 1, 2, or 3 days.

13. The method of claim 12, wherein the cardiac progenitor inducing medium comprises:
BMP4, optionally at 1-100 ng/ml,
Activin A, optionally 1-100 ng/ml,
bFGF, optionally at 1-20 ng/mL, and
CHIR-99021, optionally at 1-10 μM.

14. The method of claim 12, wherein the cardiac progenitor inducing medium comprises about 3 ng/ml BMP4 and about 2 ng/ml Activin A, optionally wherein the medium further comprises about 5 ng/ml bFGF and/or 1-10 μM CHIR-99021.

15. The method of claim 12, wherein the cardiac progenitor inducing medium comprises about 5 ng/ml BMP4 and about 4 ng/ml Activin A, optionally wherein the medium further comprises about 5 ng/ml bFGF and/or 1-10 μM CHIR-99021.

16. The method of any one of claims 12-15, wherein the embryoid bodies are obtained by incubating human pluripotent stem cells (hPSCs) in the presence of a BMP, optionally BMP2 or BMP4, and Rho-associated protein kinase (ROCK) inhibitor.

17. An AVNLPC-enriched population of cardiomyocytes generated by the method of any one of claims 1-16.

18. The population of claim 17, wherein at least 25, 30, 40, 50, 60, 70, 80, or 90% of the cells in the population are AVNLPCs.

19. A pharmaceutical composition comprising the enriched cell population of claim 17 or 18 and a pharmaceutically acceptable carrier.

20. The pharmaceutical composition of claim 19, wherein the pharmaceutically acceptable carrier is a hydrogel.

21. A method of treating a human patient in need thereof, comprising administering the pharmaceutical composition of claim 19 or 20 to the patient. 22 The enriched cell population of claim 17 or 18, or the pharmaceutical composition of claim 19 or 20, for use in treating a human patient in need thereof.

23. Use of the enriched cell population of claim 17 or 18 for the manufacture of a medicament for treating a human patient in need thereof.

24. The method, cell population or pharmaceutical for use, or use of any one of claims 21-23, wherein the human patient has atrioventricular block.
